(19) United States
(12) Patent Application Publication
(10) Pub. No.: US 2023/0310859 A1
(43) Pub. Date: Oct. 5, 2023

(54) SYSTEMS, DEVICES AND METHODS FOR PLACEMENT OF NEUROMODULATION SYSTEM VIA A SINGLE SURGICAL INCISION AND ANCHORING LEADS

(71) Applicant: SynerFuse, Inc., Eden Prairie, MN (US)

(72) Inventors: Gregory F. Molnar, Blaine, MN (US); Rohan R. Lall, Edina, MN (US); Michael Park, Excelsior, MN (US); Christopher G. Frank, New Brighton, MN (US)

(21) Appl. No.: 18/128,760

(22) Filed: Mar. 30, 2023

Related U.S. Application Data
(60) Provisional application No. 63/362,269, filed on Mar. 31, 2022.
(60) Provisional application No. 63/365,723, filed on Jun. 2, 2022.

(51) Int. Cl. A61N 1/36; A61N 1/375; A61N 1/372

(52) U.S. Cl. CPC A61N 1/36071; A61N 1/3752; A61N 1/37223; A61N 1/37235; A61N 1/3605; A61N 1/37518; A61N 1/36062

(57) ABSTRACT

Systems, devices and methods for implanting neuromodulation systems and, in some cases, spinal fixation systems using a single surgical incision and placing the system components with direct visual and physical access through an open access. Implantable pulse generators of the neuromodulation systems include lead receptacles and, in some cases, the leads may bifurcate into two leads at a proximal side of the leads. Anchors are provided that are capable of anchoring a body of a neuromodulation lead to the rod of a spinal fixation system and/or engaging bone.

### CROSS-REFERENCE TO RELATED APPLICATIONS

This application claims priority to U.S. Provisional Patent Application Ser. No. 63/365,723, titled, SYSTEMS, DEVICES AND METHODS FOR PLACEMENT OF NEUROMODULATION SYSTEM VIA A SINGLE SURGICAL INCISION, filed on Jun. 2, 2022, and further claims priority to U.S. Provisional Patent Application Ser. No. 63/362,269, titled SYSTEMS, DEVICES AND METHODS FOR PLACEMENT OF NEUROMODULATION SYSTEM VIA A SINGLE SURGICAL INCISION, filed on Mar. 31, 2022, the contents of each of which are hereby incorporated by reference.

### STATEMENT REGARDING FEDERALLY SPONSORED RESEARCH OR DEVELOPMENT

Not Applicable.

### BACKGROUND OF THE INVENTION

### Field of the Invention

The invention relates to a system and/or method for treating chronic spinal pain via implantation of a neuromodulation device and optionally in combination with a surgical procedure such as vertebral fusion, wherein the surgical procedure is conducted with open physical and visual access to the region of the spin undergoing treatment via a single incision.

### Description of the Related Art

Neuromodulation for the treatment of chronic spinal pain is a procedure that has been in use for decades. The procedure is generally prescribed to a patient only after they have gone through a spinal procedure that may involve vertebral fusion in an effort to mitigate and/or correct the supposed source of the pain. However, often such spinal procedures do not resolve the pain issues. After weeks, months and perhaps years of continued chronic pain and pain therapy through medications, including opioids, the patient may finally be prescribed neuromodulation for the treatment of chronic pain after failed back surgery.

The art does not provide single-surgical-procedure solutions that address these issues. Accordingly, it would be highly advantageous to provide a surgical method and system that enables both a spinal procedure and neuromodulation system implantation within a single procedure.

It would be further highly advantageous to enable full physical and visual access to the associated spinal treatment site for placement of the surgical fusion device and the neuromodulation system.

It would be a further advantage to provide a surgical procedure that does not require advancement of an electrical lead through a patient's anatomy to reach the ultimate location of therapeutic efficacy.

It would be a further advantage to provide implantation of the neuromodulation system during the open spinal procedure, wherein the neuromodulation system may generate electrical stimulation during and/or after the surgical procedure.

It would be a further advantage to provide implantation of the neuromodulation system via a single incision during the open spinal procedure, wherein the neuromodulation system may generate electrical stimulation during and/or after the surgical procedure.

It would be a further advantage to provide the implanted neuromodulation system as described above and for use in generating electrical stimulation only if the patient experiences pain after the surgical procedure.

Various embodiments of the present invention address, inter alia, these issues.

The figures and the detailed description which follow more particularly exemplify these and other embodiments of the invention.

### BRIEF DESCRIPTION OF THE DRAWINGS

FIG. 1 illustrates a prior art spinal neuromodulation implantation system.

FIG. 2 is an exemplary embodiment of a spinal fixation system and implantable pulse generator.

FIG. 3A illustrates an exemplary embodiment of an implantable pulse generation system.

FIG. 3B illustrates an exemplary embodiment of an implantable pulse generation system.

FIG. 3C illustrates an exemplary embodiment of an implantable pulse generation system.

FIG. 3D illustrates an exemplary embodiment of an implantable pulse generation system.

FIG. 3E illustrates an exemplary embodiment of an implantable pulse generation system.

FIG. 4A illustrates an embodiment of an exemplary neuromodulation system capable of delivering a neuromodulation therapy via multiple leads.

FIG. 4B illustrates an embodiment of an exemplary neuromodulation system capable of delivering a neuromodulation therapy via multiple leads.

FIG. 4C illustrates an embodiment of an exemplary neuromodulation system capable of delivering a neuromodulation therapy via multiple leads.

FIG. 5A illustrates an exemplary embodiment of a neuromodulation system having bifurcated leads capable of delivering a neuromodulation therapy to an anatomical target.

FIG. 5B illustrates an exemplary embodiment of a neuromodulation system having bifurcated leads capable of delivering a neuromodulation therapy to an anatomical target.

FIG. 6 illustrates an exemplary embodiment method for providing acute and chronic pain relief to a patient after a spinal fixation procedure.

FIG. 7A illustrates two exemplary embodiments of an implantable pulse generation system.

FIG. 7B illustrates an exemplary embodiment of an implantable pulse generation system.

FIG. 7C illustrates an exemplary embodiment of an implantable pulse generation system.

FIG. 8A illustrates an exemplary embodiment of a neuromodulation system of the present invention.

FIG. 8B illustrates an exemplary embodiment of an anchor for a neuromodulation system.

FIG. 9A illustrates an exemplary embodiment of an anchor use with an implantable neuromodulation system in accordance with the present invention.

FIG. 9B illustrates an exemplary embodiment of an anchor use with an implantable neuromodulation system in accordance with the present invention.

FIG. 9C illustrates an exemplary embodiment of an anchor use with an implantable neuromodulation system in accordance with the present invention.

FIG. 9D illustrates an exemplary embodiment of an anchor use with an implantable neuromodulation system in accordance with the present invention.

FIG. 9E illustrates an exemplary embodiment of an anchor use with an implantable neuromodulation system in accordance with the present invention.

FIG. 10A illustrates an exemplary embodiment of an anchor use with an implantable neuromodulation system in accordance with the present invention.

FIG. 10B illustrates an exemplary embodiment of an anchor use with an implantable neuromodulation system in accordance with the present invention.

FIG. 10C illustrates an exemplary embodiment of an anchor use with an implantable neuromodulation system in accordance with the present invention.

FIG. 10D illustrates an exemplary embodiment of an anchor use with an implantable neuromodulation system in accordance with the present invention.

FIG. 11 illustrates an exemplary embodiment of an implantable pulse generator in accordance with the present invention

FIG. 12 illustrates an exploded view of the embodiment of the implantable pulse generator of FIG. 12.

FIG. 13 illustrates a perspective view of an exemplary embodiment of a charging system for charging the battery of the implantable pulse generator.

FIG. 14 illustrates an exemplary embodiment of an external programmer interface for programming the implantable pulse generator via a programmer interface.

### DETAILED DESCRIPTION OF THE INVENTION

This application incorporates by reference the entire contents of U.S. patent application Ser. No. 16/519,512, titled METHODS AND SYSTEMS FOR IMPLANTING A NEUROMODULATION SYSTEM AT A SURGICALLY OPEN SPINAL TREATMENT SITE, filed on Jul. 23, 2019 and issued as U.S. Pat. No. 11,247,046 on Feb. 15, 2022.

This application also incorporates by reference the entire contents of U.S. Provisional Application 62/702,867, filed Jul. 24, 2018 and entitled METHOD FOR IMPLANTING A NEUROMODULATION SYSTEM AT A SPINAL TREATMENT SITE.

This application further incorporates by reference the entire contents of U.S. patent application Ser. No. 16/519,320, titled METHODS AND SYSTEMS FOR IMPLANTING A NEUROMODULATION SYSTEM AND A SPINAL FIXATION SYSTEM AT A SURGICALLY OPEN SPINAL TREATMENT SITE, filed on Jul. 23, 2019 and issued as U.S. Pat. No. 10,675,458 on Jun. 9, 2020.

Further, this application incorporates by reference the entire contents of U.S. Provisional Application 62/702,867, filed Jul. 24, 2018 and entitled METHOD FOR IMPLANTING A NEUROMODULATION SYSTEM AT A SPINAL TREATMENT SITE.

Generally, various embodiments of the present invention are based upon the premise that many patients who suffer from chronic back pain, such as those who suffer for a long enough period of time or due to the severity of their particular condition, are also separately suffering from neuropathic pain that cannot be corrected by spinal surgery. In such cases, it is a misnomer to say that a patient is suffering from “failed back surgery” but more accurately that the back surgery simply does not address the neuropathic pain that may have been in place prior to the back surgery.

The present invention provides a method for combining the implantation of a spinal treatment device with the implantation of a neuromodulation device, or at least a neuromodulation lead of a neuromodulation device, into a single combination procedure performed at the spinal treatment site. The present invention thus provides the potential to treat both back stabilization issues and neuropathic pain issues in a single procedure, with the additional benefit of minimizing the amount of pain medications, including opioids and other pain medications that a patient may otherwise require to manage chronic back pain.

The present invention further provides a system, device and method for treating chronic neuropathic pain either alone or in combination with another spinal procedure.

Currently available spinal cord stimulation and slash or neural modulation systems generally include an implantable pulse generator having a battery and the battery coupled to a circuit board for a programming and further having a number of electrical connectors for coupling the implantable pulse generator with one or more leads.

A typical implantable pulse generators for spinal cord stimulation have a header with two receptacles for coupling with a corresponding pair of leads. One example of such system is the Medtronic Intellis™ SCS system that provides an implantable pulse generator having a height of 57.1 millimeters and a width of 47.2 millimeters and a volume of 13.9 cubic centimeters.

This prior art system includes a header with receptacles for coupling to two leads of eight contacts each, the contacts being embedded within the header. This system therefore has a limitation that only allows for the use of two leads in combination with the implantable pulse generator.

Another prior art neural modulation device is the Abbott Proclaim™ implantable pulse generator which is marketed for the neural modulation of the dorsal root ganglia (DRG). This pulse generator has dimensions of a height of 61 millimeters a width of 49.5 millimeters and a depth of 1.34 millimeters for a total volume of 40 cubic centimeters. This implantable pulse generator of the prior art allows for a header with four lead receptacles each receptacle having 8 contacts before he total love 32 contacts.

An objective of the present invention is to provide a neural modulation system that allows for the use of up to four leads couplable to an implantable pulse generator where the leads and the implantable pulse generator are all implantable at a single surgical incision.

It is further an objective of the present invention to provide such a neuro modulation system that is implantable at a spinal treatment site where in a spinal fixation procedure is being performed and we are in such neural modulation system is implantable via the same surgical incision as the spinal fixation procedure incision.

The shortcoming of the above described prior art is that the smaller volume devices such as the Medtronic device discussed above do not accommodate four leads, and even so are of a form factor that is greater than 15 cubic centimeters. Furthermore, devices that are capable of accommodating four leads, such as the Proclaim™ device described above, have an even larger form factor that is greater than 20 cubic centimeters.

It would therefore be advantageous to have an implantable pulse generator of a form factor small enough to be implanted in the incision of a spinal treatment site and that is able to accommodate more than two leads, preferably 4 leads or more.

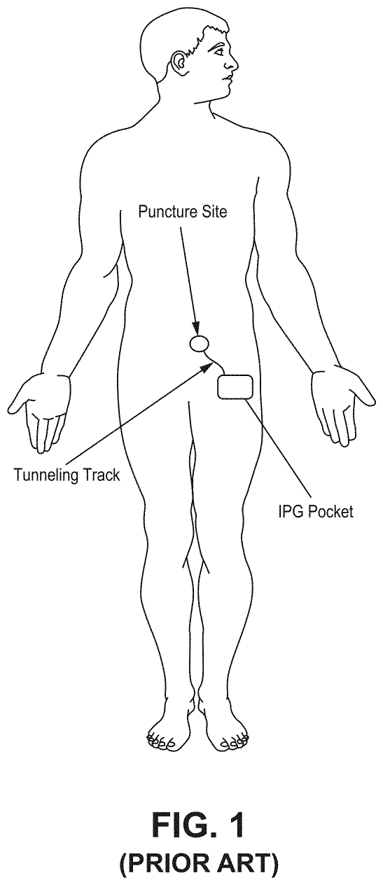

Moreover, as shown in FIG. 1, both of the described prior art devices require two separate incisions in order to fully implant the neuro modulation system. The leads are generally implanted via a puncture entry site and the implantable pulse generator is generally implanted by the creation of a pocket for the implantable pulse generator. The leads are then tunneled from the puncture site to the implantable pulse generator pocket.

It would therefore further be advantageous to have an implantable neural modulation system that is entirely implantable via a single surgical incision. Furthermore, it would be advantageous to be able to implant the neuro modulation system at the same surgical incision as a spinal fixation procedure.

We turn now to discuss an embodiment of a method of implanting a neuromodulation system in a surgical bed via a single surgical incision either alone or in combination with a spinal fixation procedure. First, two or more targeted vertebral levels may be identified for vertebral stabilization. One or more associated targeted spinal levels may be identified for neuro modulation stimulation. A surgical incision is performed to create an open access into the resulting spinal treatment site to provide full visual and physical access to the identified targeted vertebral levels and the at least one identified targeted spinal level. The identified vertebral levels may be stabilized and then the neuro modulation system is implanted via the same open access, created with a single surgical incision, to the spinal treatment site.

A first lead or pair of leads may be disposed such that the electrodes at a distal end of the leads are placed in therapeutic proximity to a dorsal root ganglion or other nerve target. This first set of leads may be placed near the same dorsal root ganglia at the same level as the identified targeted spinal level. A second set of one or more leads may be placed at an adjacent spinal level either above or below the targeted spinal level. Preferably, the adjacent spinal level will be the spinal level below the targeted spinal level of the first one or more set of leads in order to provide optimal coverage and therapy for the treatment of neuropathic spinal pain. Once the leads are placed at the corresponding dorsal root ganglia and corresponding spinal levels the implantable pulse generator is implanted in the surgical bed within or approximately within the plenum defined by the spinal fixation procedure. The proximal ends of the leads are then coupled to the corresponding receptacles of the implantable pulse generator.

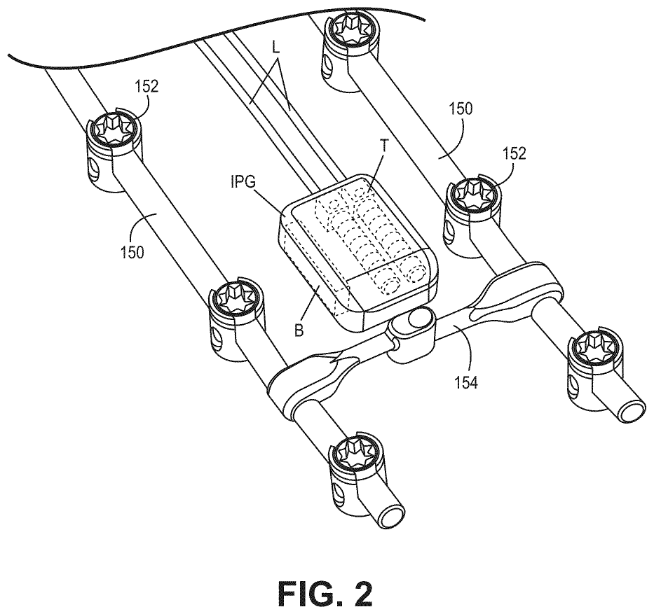

FIG. 2 illustrates an exemplary system 100 comprising vertebral stabilization and implanted neuromodulation system that is configured to be implanted within the open access formed by a single surgical incision as discussed above. System 100 comprises two spaced-apart stabilization rods 150 that are secured using anchors 152. A spanning member 454 may be used to stabilize the two rods 150 and ensure proper spacing is maintained. An IPG may be placed between the two rods 150, the IPR comprising a top T and a bottom B, with two or more leads L electrically connected to the IPG and extending away to the target(s) of interest, such as a DRG.

Next, a method is presented for implanting a neuromodulation system via a single incision either alone and/or in combination with a spinal fixation procedure. In this embodiment of the present invention, a first set of one or more leads is implanted over a nerve target, such as the DRG, at the targeted spinal level. And, optionally, a second set of one or more leads may be implantable at an adjacent spinal level over a nerve target such as a DRG or other nerve target at such adjacent spinal level.

Prior to implanting the implantable pulse generator, the proximal ends of the implanted leads may be fed through the sub-fascial level and the implantable pulse generator may be implanted between the subfascial level and the outer derma layer, more generally, the fatty tissue layer between the outer dermal layer and the sub fascia.

The proximal ends of the leads may then be inserted into the corresponding receptacles of the header of the implantable pulse generator. The surgical incision is then closed and a neural modulation therapy may be delivered to the nerve targets via the implantable pulse generator.

The implantable pulse generator can be positioned any tissue location accessible via the surgical incision, including the midline of the spine, fully offset from the midline of the spine, at a spinal level above or below the target spinal level as determined by a surgeon and/or preferable by the nature of the particular procedure.

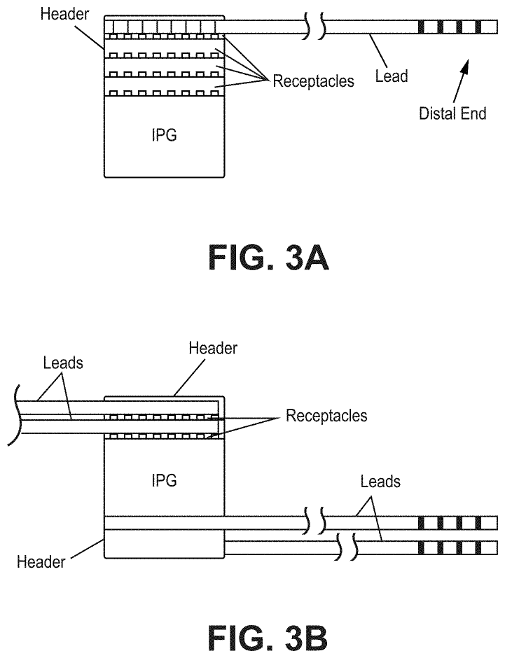

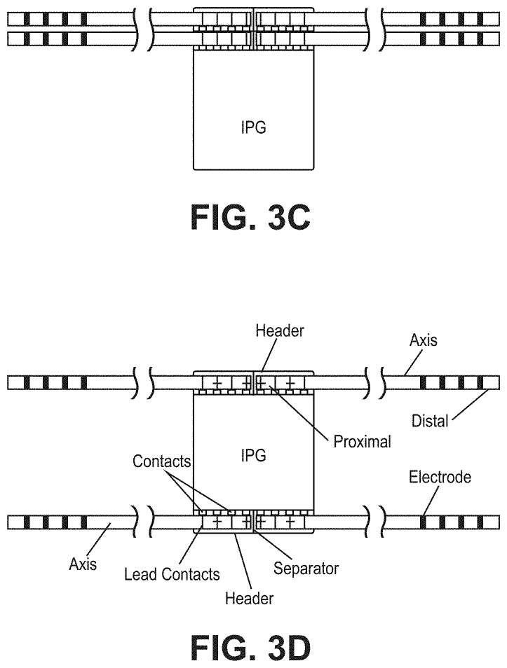

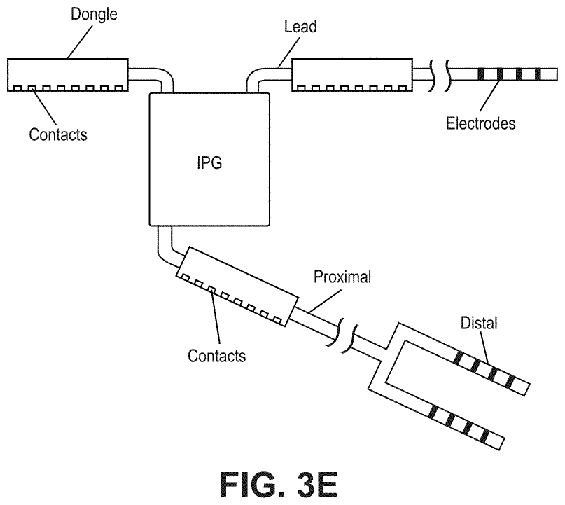

FIGS. 3A-3E illustrate embodiments of neuromodulation systems capable of delivering a neuromodulation therapy via multiple, i.e., more than two, leads.

FIG. 3A illustrates an exemplary implantable pulse generator IPG with a header capable of receiving one or more leads therein. In the embodiment shown, the header has four receptacles and is a stacked header in that each of the receptacles is stacked on a previous receptacle forming a plane extending from the narrow depth dimension of the implantable pulse generator. Providing four receptacles allows for the first set of leads to be positioned on a first pair of nerve targets at a first spinal level and a second pair of leads to be positioned on a second pair of nerve targets.

FIG. 3B illustrates an exemplary implantable pulse generator with a header capable of receiving four leads where in a first header is on one side of the implantable pulse generator and a second header he's on the opposing side of the implantable pulse generator the combination allowing for a thin profile in the depth dimension of the implantable pulse generator and to allow for each separate pair of leads to be received within a separate spaced set of receptacles.

FIG. 3C illustrates an implantable pulse generator with a first header that has an axial dimension allowing for two separate lead receptacles along the same axis for receiving a first set of leads and the second set are having an axial dimension allowing for two additional separate lead receptacle along the second axis dimension.

FIG. 3D illustrates an alternative embodiment of the implantable pulse generator from FIG. 3C wherein the first header and second header are on opposing sides of the implantable pulse generator.

It is contemplated that various numbers of leads and combinations of header arrangements may be utilized in accordance with the present invention. In a preferred embodiment of the present invention the implantable pulse generator is capable of receiving and or electrically coupling with four leads each of the leads having four electrodes and therefore a corresponding for channel coupling at the header receptacle for each lead. As a result, the implantable pulse generator would have a total of 16 channels, four channels for each lead receptacle for coupling with a corresponding coupling element of the leads.

It is contemplated that it is within the scope and spirit of the invention that any number of contacts can be utilized in any number of lead receptacles including different lead receptacles having different numbers of contacts therein.

FIG. 3E illustrates an alternative embodiment of an implantable pulse generator IPG having one or more dongles extending from the IPG, each of the dongles capable of receiving a lead therein and each of the dongles having a set of contacts for coupling to a corresponding set of lead contacts in order to provide an electrical coupling to the lead and to deliver a neuro modulation therapy to the distal electrodes of the leads.

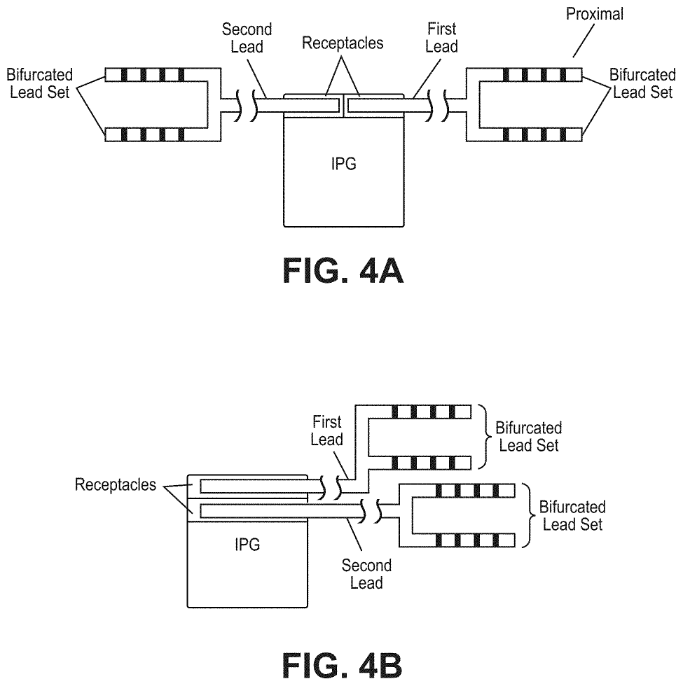

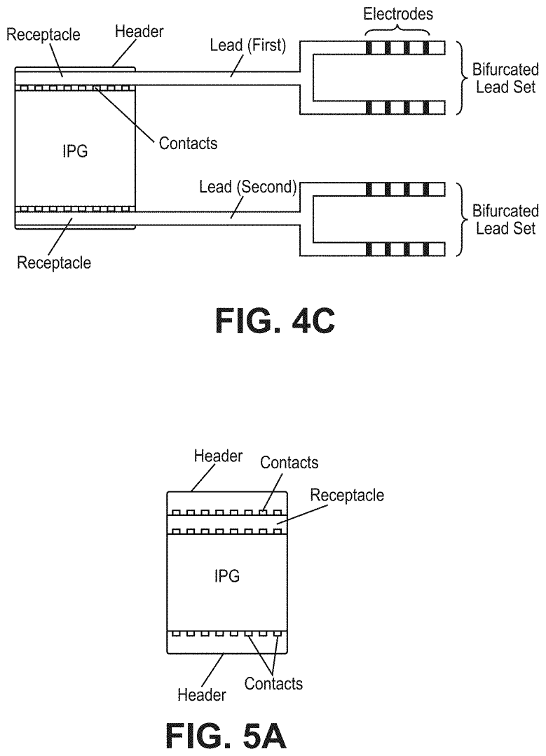

FIGS. 4A-4C are embodiments of exemplary neuromodulation systems having bifurcated leads capable of delivering a neuromodulation therapy to an anatomical target.

As shown, the header size can be reduced by replacing two lead receptacles with a single lead receptacle having more closely spaced contacts and slash or fewer numbers of contacts. By way of example, FIG. 4A illustrates an IPG having two lead receptacles wherein each of these separate lead receptacles may have contacts that are closely spaced together so as to provide for a small form factor of the implantable pulse generator. The proximal end of the lead bifurcates at a location distal from the header such that four of the electrical contact wires are extended to one of the bifurcated lead to distal ends and the other four of the electrical contact wires extend to the other of the bifurcated lead distal end for a first pair of distal lead portions. A second lead is likewise coupled to the second header receptacle area and is likewise bifurcated to provide for and eat contact coupling at the header, the proximal portion of the bifurcated lead, and then a bifurcate lead portion having a first bifurcated lead having for example, 4 electrodes and a second bifurcated portion having 4 electrodes.

FIG. 4B illustrates an alternative embodiment wherein the two receptacles are stacked, one above the other and adjacent each other, to provide connection for first and second leads, each of which are bifurcated as discussed above.

FIG. 4C is a variation of FIG. 4B, wherein the two receptacles are spaced apart, one near an upper surface of the IPG and another near a lower surface of the IPG. Each receptacle thus provides connection for first and second leads, each of which are bifurcated as described above.

Any number of variations of lead header arrangements and designs may be utilized in combination with any combination of bifurcated or unitary leads being electrically coupled to the corresponding headers.

The various embodiments of an implantable pulse generator provided herein are intended to allow for a small form factor or volume of the implantable pulse generator so as to enable placement of the IPG to the spinal treatment site, whether alone or in combination with a spinal fixation procedure, and further allowing for the IPG to be implanted in the same incision as the leads.

In a preferred embodiment the one or more leads, preferably four leads, results in an IPG having a total volume of less than about 12 cubic centimeters, or preferably a volume of less than about 10 cubic centimeters, or more preferably a volume of less than about 8 cubic centimeters, and more preferably a volume of less than about 5 cubic centimeters.

Such an IPG may be a rechargeable implantable pulse generator system or a primary cell system depending upon the energy requirements and recharge requirements of the particular procedure.

In a preferred embodiment, where the first set of one or two leads are implanted on a first set of dorsal root ganglia at a first spinal level and a second set of one or two leads are implanted at a second spinal level on a corresponding second set of dorsal root ganglia, each of the four implanted leads having four electrodes, it is anticipated that the small form factor of the implantable pulse generator is obtainable in a number of configurations as have been described here in.

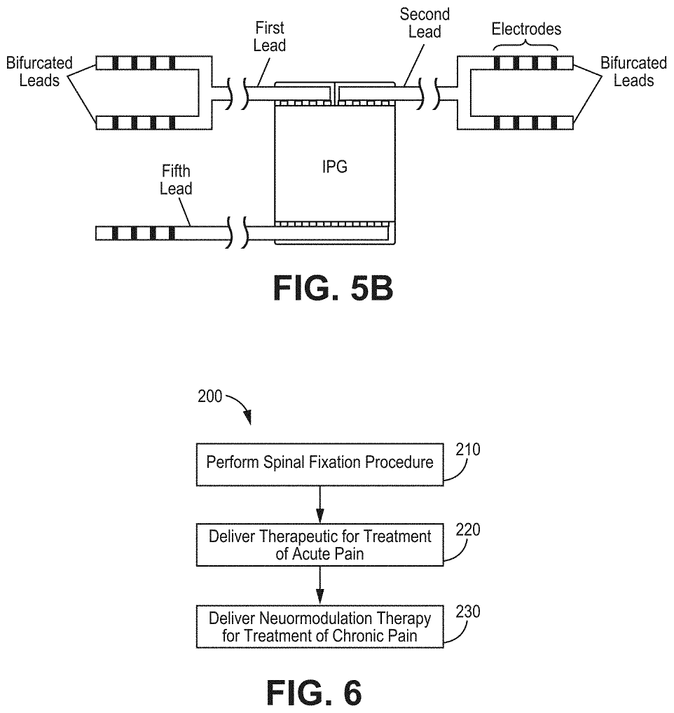

FIGS. 5A and 5B illustrate exemplary embodiments of neuromodulation system and lead combinations in accordance with the present invention. Where in the in above examples have largely focused on the nerve target to be in the dorsal root ganglia it is anticipated within the scope of the invention that additional nerve targets may be targeted via the present invention also or alternatively additional anatomical targets may be targeted in accordance with the present invention. FIGS. 6A and 6B thus illustrate an IPG having four leads that may target corresponding dorsal root ganglia and then a fifth lead with a separate header receptacle for targeting a different anatomical target including but not limited to the spinal cord, bone for bone growth stimulation, peripheral nerves, muscles and other anatomical targets and tissues. Likewise, any and all of the leads may target different anatomical targets in accordance with the present invention.

FIG. 6 is a flow chart of an exemplary method for providing acute and chronic pain relief to a patient after a spinal fixation procedure. The method 200 for treating pain associated with a patient's spinal column includes performing a spinal fixation procedure 210 which may comprise:
identifying two or more targeted vertebral levels for vertebral stabilization;
identifying one or more targeted spinal levels for neuromodulation stimulation;
creating an open access into the resulting spinal treatment site to provide full visual and physical access to the identified targeted vertebral levels and the at least one identified targeted spinal level;
stabilizing the identified vertebral levels; and
may further comprise implanting a neuromodulation system at the spinal treatment site, via the open access for providing long term pain relief.

Next, a pain alleviating drug may be dispensed or administered to the spinal treatment site via the open access for providing acute post-vertebral stabilization pain relief at step 220. The pain alleviating drug may comprise a long-acting non-opioid drug such as a long acting lidocaine or Novocain such as Exparel and/or similar long acting localized pain therapy therapeutics. Such a therapeutic, in accordance with the present invention is capable of providing acute pain relief for up to at least 24 hours or for up to 48 hours or for up to 72 hours or for up to one week post procedure. As such, the therapeutic would provide pain relief for the acute pain of a patient resulting from the spinal procedure and the neuromodulation system would provide pain relief from chronic and neuropathic pain resulting from ongoing spinal pain. Finally, neuromodulation therapy may be delivered at step 230.

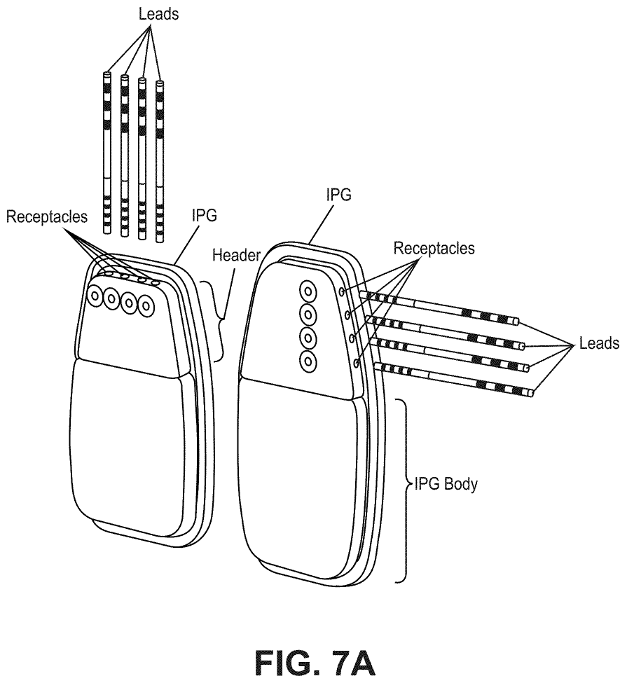

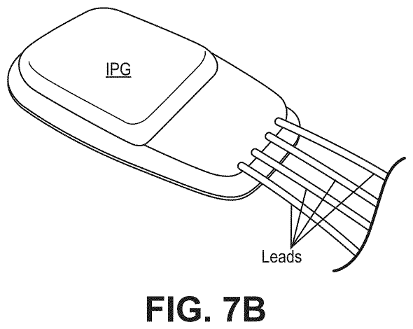

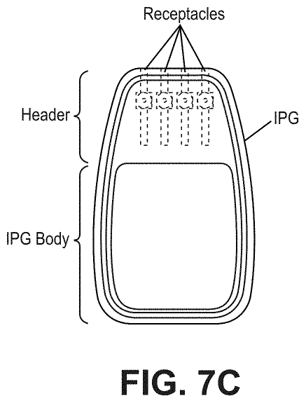

FIGS. 7A-7C illustrate an alternative embodiment of an implantable pulse generator for use in accordance with the present invention both the system and the methods thereof. Such implantable pulse generators may have a volume of less than about 15 cubic centimeters, less than about 12 cubic centimeters, less than about 10 cubic centimeters, less than about 8 cubic centimeters, less than about 5 cubic centimeters, in accordance with the methods and systems of the present invention in the objectives of a user of the present invention and methods and/or of a patient being implanted with the present inventions and or methods.

Traditional implantable pulse generators have an implantable pulse generator body, making up the bottom portion of the illustrations shown in FIGS. 7A-7C and then traditional implantable pulse generators have a header wherein the header has one or more receptacles for receiving a proximal end of an electrical stimulation lead therein. Each of the one or more receptacles having an axis that is parallel to the proximate edge of the implantable pulse generator to which it is connected. In the present embodiment, as shown on the IPG illustrated on the left side of the page with reference to FIG. 7A, the header is oriented in a perpendicular or orthogonal relationship with respect to the body of the implantable pulse generator. Said differently, the header of the present invention described in this figure has one or more receptacles wherein the receptacles have an axis for receiving a proximal end of one or more who responding electrical stimulation leads, such access being arranged perpendicular to the body of the implantable pulse generator.

FIG. 7B is an illustration of an exemplary embodiment of the present invention from FIG. 7A with four leads. FIG. 7C is an alternative illustration of the embodiment of the present invention discussed with reference to FIGS. 7A and 7B, showing the header having four lead receptacles for receiving the proximal end of four leads, the lead receptacles of the header having an access that extends perpendicular to the proximate edge of the implantable pulse generator. Within the header, one or more contacts exists within each lead receptacle, the present invention showing four contacts in each lead receptacle for a total of 16 channels. Each lead receptacle further having a means for securing the lead within the receptacle for providing the electrical coupling and maintaining the electrical coupling of the header contacts to the corresponding contacts of the proximal end of the lead for each lead receptacle.

An exemplary embodiment of the method and system of the present invention wherein a neuromodulation system is implanted at a spinal fixation site and wherein the leads are anchored within the spinal fixation site is now described.

As discussed above, an IPG may be provided with connection for four leads implanted at the spinal fixation site wherein a first and second fixation rod are implanted at a first spinal level and a first set of dorsal root ganglia are anatomically located at that spinal level, also shown is a second set of dorsal root ganglia at a second anatomical level, in the present embodiment the second anatomical level is the level below the fixation level however it is understood that any alternative spinal level including proximate or non-proximate spinal levels can be accessed within the spirit of the present invention.

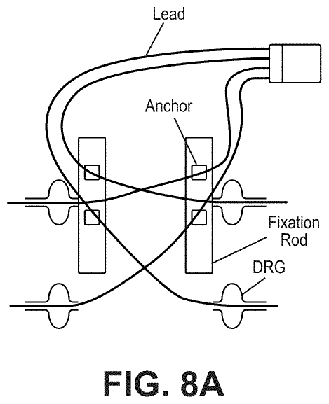

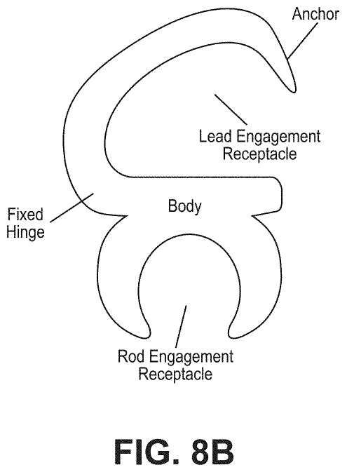

As discussed above, a set of four electrical stimulation leads may be implanted, however it is understood that any number of one or more leads may be implanted at the spinal treatment site, including but not limited to a single lead at a single DRG at a single spinal level or one or more leads at each of the identified spinal levels. By way of example only, a first set of leads is implanted at the first spinal level at which the fixation rods are positioned and a second set of leads are implanted at the second spinal level. The leads follow a lead path wherein each lead approaches the associated target dorsal root ganglia bypassing either over or under both the nearest fixation rod and the more distal fixation rod such that at least one of the one or more leads is anchored at the distal fixation rod as compared to the targets dorsal root ganglia on which the distal portion of the electrical stimulation lead is placed for therapeutic delivery. In the present embodiment, each of the four leads may be anchored to a corresponding fixation rod such that the lead is anchored to the fixation rod most distal to the target dorsal root ganglia on which the lead is placed as shown in FIG. 8A.

The proximal end of each lead may then coupled to the implantable pulse generator via the header, the implantable pulse generator and header may be in accordance with any of the embodiments described herein above.

FIG. 8B is an anchor in accordance with the present invention, particularly unicorns with the invention described in FIG. 8A. The anchor has an anchor body and extending from one portion of the anchor body is a rod engagement receptacle wherein the anchor may be friction fit onto a fixation rod, such as described and illustrated in FIG. 2, via the rod engagement receptacle. On an opposing portion of the lead body is a lead engagement receptacle wherein the lead engagement receptacle is capable of receiving a portion of a lead body therein and maintaining the lead body within the lead engagement portion of the anchor. As shown comment lead engagement portion may be a fixed hinge receptacle where in a lead may be fitted into the lead engagement portion by opening the receptacle we're in the league engagement portion will then return to its more constricting state and thereby engage and anchor the lead therein.

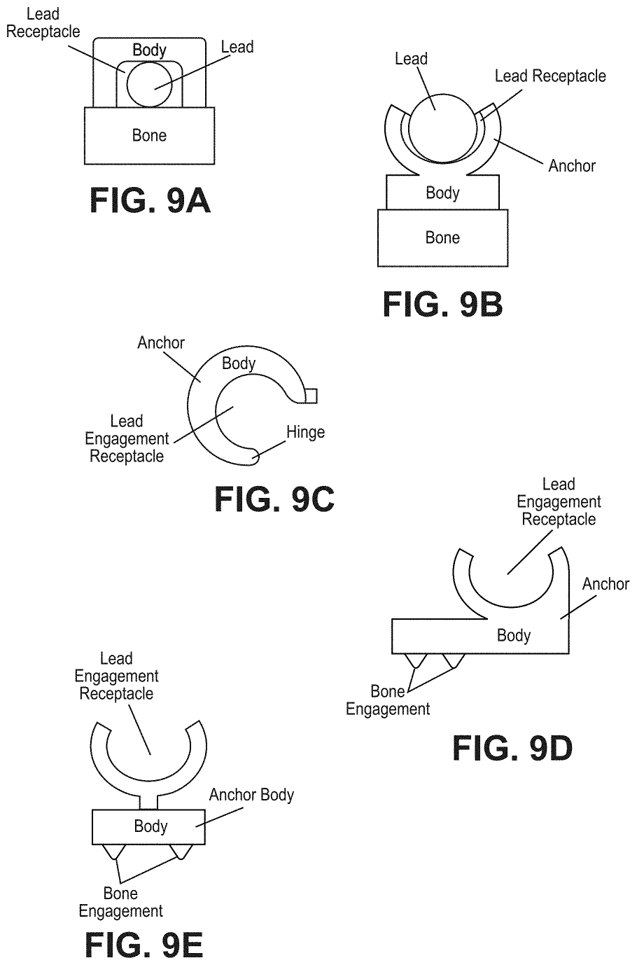

FIGS. 9A-9E illustrate a number of alternative embodiments to an anchor for use alone or in combination with the present methods and systems of the current invention and its various embodiments. The anchors have an anchor body and the anchor engagement portion for engaging one or more of the fixation rod or bone or tissue of a patient. The anchors further have a lead engagement receptacle or portion for engaging with a lead such that any of the anchors or combinations of anchor engagement portions and or anchor lead. Lead receptacle or engagement portions may be combined such that a lead may be anchored to a fixation rod or pedicle screw or a crossbar or a tissue or a bone and the lead baby engaged using any of the various lead engagement methods illustrated and described herein.

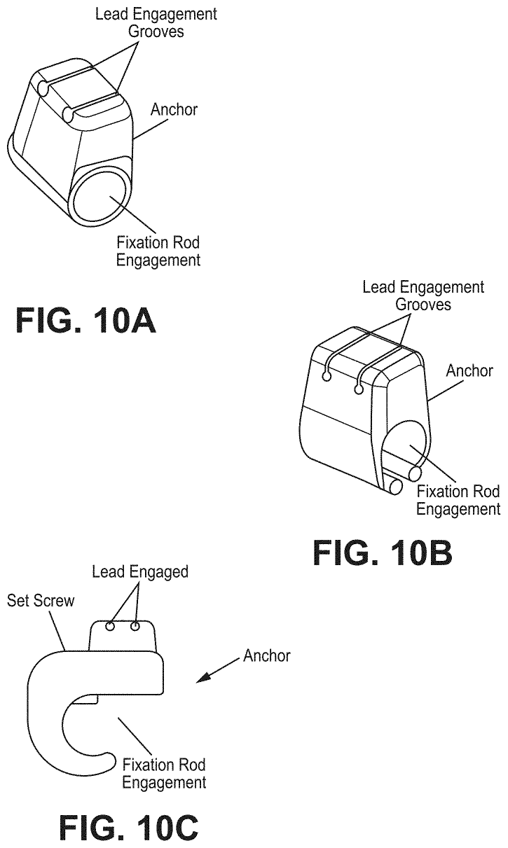

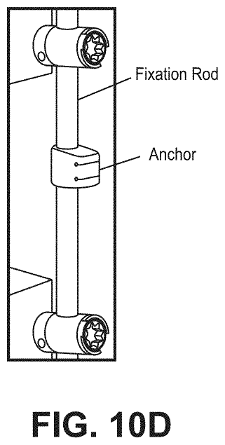

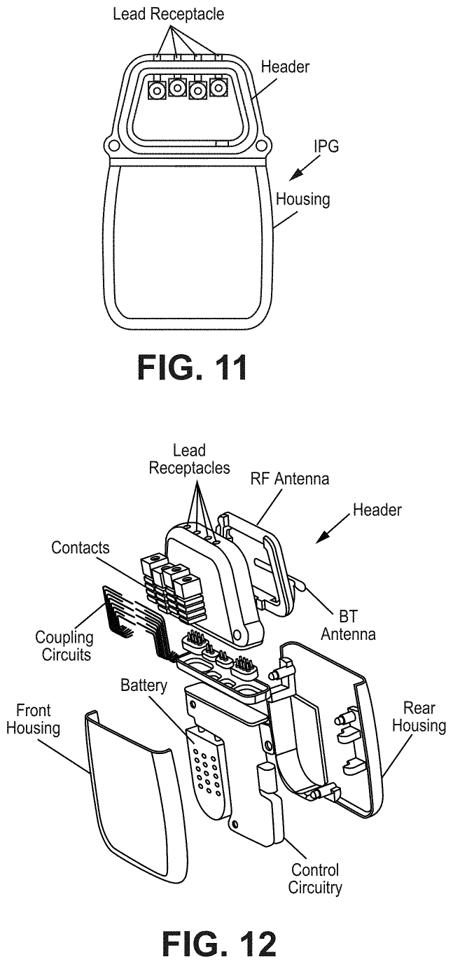

FIGS. 10A-10C illustrate additional alternative embodiments of an anchor in accordance with the present invention. FIG. 10A FIG. 11d illustrates an example of an anchor embodiment engaged on a rod in accordance with the present invention, whereas any of the various anchors described herein may be so anchored.

FIG. 10A illustrates an anchor having a fixation rod engagement portion defining a receptacle for sliding the rod engagement portion on a fixation rod prior to surgical placement. The anchor further has one or more lead engagement grooves that define a lead engagement portion. The lead engagement grooves are formed of a plastic material capable of frictionally receiving and engaging a lead when placed in the groove to prevent or minimize axial migration of the lead body engaged therein.

FIG. 10B shows an alternative embodiment of that shown in FIG. 11a wherein the fixation rod engaging portion is a generally C-shaped snap on style wherein the rod engaging portion can be used to snap the anchor on to the rod after the rod has been surgically placed. The rod engaging portion may be a plastic material that frictionally engages the rod and prevents rotation of the anchor with respect to the rod once engaged on the rod. Suitable plastic materials include any moldable or formable materials having biocompatible properties, including but not limited to silicon, PEEK and the like.

FIG. 10C shows an alternative embodiment of an anchor from that shown in FIGS. 10A and 10B, wherein the fixation rod engaging portion is a rigid material such as titanium or stainless steel or any other such rigid material wherein the anchor engaging portion is positioned on the rod after insertion into the surgical location and a set screw is used to fasten the anchor engaging portion and prevent rotation of the anchor with respect to the rod once the set screw has been tightened.

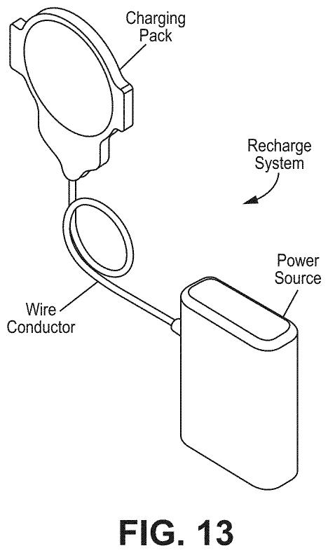

FIGS. 11 and 12 (exploded view) illustrate an exemplary embodiment of an implantable pulse generator of the present invention wherein the recharge antennae of the implantable pulse generator extends around a perimeter of the header, the antennae allowing for wireless recharging of the battery of the implantable pulse generator when implanted in a patient via radiofrequency from an external charger. An exemplary recharging system comprising a charging puck electrically connected with a power source is shown in FIG. 13.

The implantable pulse generator may be surgically positioned within the body of the patient such that the recharge antennae is about 1-2 cm, or 2-4 cm or 4-8 cm or less than 8 cm from the surface of the patient's skin in order to allow for wireless recharging via the antennae. The implantable pulse generator may be implanted at a position that is offset from the centerline of the patient's spinal cord but placed via the same single surgical incision as the spinal procedure allowing for a single incision for performance of the spinal fixation procedure and implantation of the neuromodulation device including one or more of the implantable pulse generator the stimulation leads and the one or more lead anchors for anchoring the leads to the spinal fixation device and/or to anatomical tissue within the surgical site.

FIG. 12 is an exploded view of the implantable pulse generator showing the recharge antennae, the programmer antennae, the control circuitry including one or more of the battery, the folded flexible circuit board enabling for the small form factor, the connectors extending from the circuit board to the one or more of the lead receptacles, recharge antennae and controller antennae, and each of the lead receptacles having one or more contacts each of which being coupled to the control circuitry for providing an electrical signal from the control circuitry to the contacts in accordance with a neuromodulation/electrical stimulation program. The control circuitry being housed within the opposing sealed housing panels and the header being sealably connected to a side of the housing having connector feedthroughs for coupling the lead contacts to the control circuitry.

As noted above, FIG. 13 illustrates an exemplary external charging system for externally charging the battery of an implanted pulse generator. The external charging system generally comprises a power source, a charging puck and a wire coupling the power from the power source to the charging puck. The charging puck delivers a charging signal in the form of radiofrequency through the skin and tissue levels of the patient whereas the radiofrequency antennae of the implantable pulse generator receives the charging signal and utilizes the charging signal to power the battery via control circuitry of the implantable pulse generator as is commonly known in the art. The power source may have one or more user controls and indicators, including but not limited to a power on/off button, a battery charge indicator indicating the charge status of the implantable pulse generator during the course of the charging process, a signal strength indicator indicating the strength of the radiofrequency connection between the charger and the implantable pulse generator.

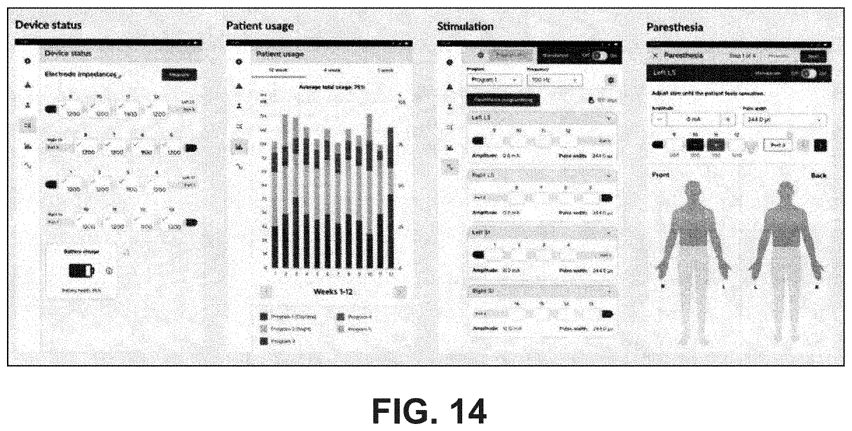

FIG. 14 is an exemplary embodiment of an external programmer interface for programming the implantable pulse generator. An external programmer may be configured to communicate wirelessly with the implanted pulse generator such as via Bluetooth or other wireless signals, as commonly known in the art, such that the communicated between the programmer and implantable pulse generator is performed via the programmer antennae within the implantable pulse generator and corresponding communicating antennae of the programmer. The programmer enables the programming of one or more therapies to be delivered by the implantable pulse generator including but not limited to programming of the frequency, amplitude, waveform and variations thereof to be delivered from the implantable pulse generator to the one or more electrodes of the distal portion of the implanted leads. By way of example, a user may select which electrode or electrodes of each of the one or more leads to be active and to determine which neuromodulation therapy parameters to deliver to the programmed electrode or electrodes including but not limited to delivering one or more neuromodulation therapies to one or more electrodes on one or more leads located at one or more anatomical targets.

It is understood that various combinations and modifications of the embodiments disclosed herein may be made in accordance with the scope and spirit of the present invention.

1. An implantable pulse generator capable of providing an electrical stimulation therapy to an anatomical target, the implantable pulse generator comprising:
a housing body for housing electronic circuitry of the implantable pulse generator, said housing forming a sealed plenum for housing said electronic circuitry and capable of maintaining separation of said electronic circuitry from biological contamination when said implantable pulse generator is implanted in a biological structure; and
a header body forming a sealed connection with a first side of said housing body, said header body having one or more receptacles comprising one or more electrical contacts, said header capable of receiving a proximal end of a lead therein and capable of delivering an electrical signal from the electrical circuitry to the one or more electrical contacts within the one or more receptacles wherein the one or more receptacles define an elongate axis extending therethrough, said axis of said one or more receptacles extending in a direction perpendicular to a plane defined by the first side of said housing body.

2. The implantable pulse generator of claim 1 wherein the header comprises two or more receptacles, each of the receptacles configured to receive a proximal end of a lead therein and having one or more electrical contacts positioned therein in electrical communication with said electrical circuitry and capable of delivering said electrical signal from said electrical circuitry to said electrical contacts.

3. The implantable pulse generator of claim 1 wherein the header comprises at least four receptacles, each of the receptacles configured to receive a proximal end of a lead therein and having one or more electrical contacts positioned therein in electrical communication with said electrical circuitry and capable of delivering said electrical signal from said electrical circuitry to said electrical contacts.

4. The implantable pulse generator of claim 2, further comprising at least one lead having a proximal end having at least one electrical contact at a proximal end thereof, said at least one electrical contact being positioned in electrical communication with corresponding said electrical contact within one of said receptacles when said proximal end of said at least one lead is positioned within said receptacle; said lead having at least one therapy delivery electrode at a distal end thereof in electrical communication with said at least one electrical contact.

5. The implantable pulse generator of claim 4, further comprising an antennae for receiving a communication from an external programmer, said antennae capable of receiving a programming signal from said external programmer and delivering said programming signal to said electrical circuitry and thereby delivering a programmed therapy in accordance with the received programming signal.

6. The implantable pulse generator of claim 5, wherein the programmed therapy comprises causing a predetermined electrical stimulation therapy output to be delivered to the therapy electrode of the distal end of the lead via the electrical circuitry of the implantable pulse generator.

7. A method for delivering a neuromodulation therapy to an anatomical target via a single incision procedure, said method comprising:
creating a single incision wherein said single incision provides direct physical and visual access to the anatomical target;
implanting via said single incision a distal portion of a lead comprising one or more distal electrodes in therapeutic proximity to said anatomical target, said lead further comprising a proximal portion having one or more proximal contacts each electrically coupled to a corresponding one or more distal electrode;
implanting via said single incision an implantable pulse generator having a housing for housing electrical circuitry capable of delivering a neuromodulation therapy and further comprising a header having a receptacle capable of receiving said proximal end of said lead wherein said receptacle has a receptacle contact for engaging a corresponding proximal contact of said lead, wherein said receptacle contact is electrically coupled to said electrical circuitry, said header being connected to said housing at a first side of said housing and said receptacle extending along an axis that extends perpendicular to a plane defined by said first side of said housing;
inserting the proximal portion of said lead into said receptacle such that proximal portion of said lead is electrically coupled to said receptacle contact;
closing said single incision; and
delivering a neuromodulation therapy to the anatomical target by delivering an electrical signal from said electrical circuitry to said one or more distal electrodes.

8. The method of claim 7 wherein said lead is anchored to anatomical tissue via an anchor.

9. The method of claim 7 wherein said lead is anchored to anatomical tissue via fibrin glue.

10. The method according to claim 6 wherein said single incision procedure further comprises implanting of a spinal fixation device.

11. The method of claim 10 wherein said lead is anchored to said spinal fixation device by an anchor.

12. The method of claim 10 wherein said lead is anchored to said spinal fixation device by fibrin glue.

13. The method of claim 7 wherein the anatomical target is a dorsal root ganglion.

14. An anchor capable of anchoring a body of a neuromodulation lead to the rod of a spinal fixation system, said anchor comprising:
a rod engagement element comprising a c-shaped receptacle having opposing arms capable of retractably receiving a rod therein and frictionally closing over an outer surface of said rod; and
a lead engagement element comprising a slot made of a semi-flexible material capable of receiving a portion of a lead body therein and frictionally maintaining said lead body therein.

15. An anchor capable of anchoring a body of a neuromodulation lead to the rod of a spinal fixation system, said anchor comprising:
a rod engagement element comprising a c-shaped receptacle having opposing arms capable of retractably receiving a rod therein by tightening said arms with respect to each other via a set screw; and
a lead engagement element comprising a slot made of a semi-flexible material capable of receiving a portion of a lead body therein and frictionally maintaining said lead body therein.
